SEQUENCE LISTING (9 sequences)

SEQ ID NO: 1 - DNA, 50 bp, synthetic construct
  misc_feature 1..50: note = Synthetic proboe
  source 1..50: mol_type = other DNA
actaacaaatatcttaaattaaaacctaaatccataacacaaaaccacac

SEQ ID NO: 2 - DNA, 50 bp, synthetic construct; identical to SEQ ID NO: 1
  misc_feature 1..50: note = Synthetic proboe
  source 1..50: mol_type = other DNA

SEQ ID NO: 3 - DNA, 50 bp, synthetic construct
  misc_feature 1..50: note = Synthetic proboe
  source 1..50: mol_type = other DNA
raaactaattatcttaactaaccctataaatactaactaaacraccracc

SEQ ID NO: 4 - DNA, 50 bp, synthetic construct
  misc_feature 1..50: note = Synthetic proboe
  source 1..50: mol_type = other DNA
caacttaactaacatataaccatataaatctaaaccaaaccctttcaacc

SEQ ID NO: 5 - DNA, 50 bp, synthetic construct
  misc_feature 1..50: note = Synthetic proboe
  source 1..50: mol_type = other DNA
cctacctaaataaactcaatttaaaaaccaaaactaaacctaacaaaacc

SEQ ID NO: 6 - DNA, 50 bp, synthetic construct
  misc_feature 1..50: note = Synthetic proboe
  source 1..50: mol_type = other DNA
ctccttctaatatacaacaaaaacactactcacaaaaacacacaaaccrc

SEQ ID NO: 7 - DNA, 50 bp, synthetic construct
  misc_feature 1..50: note = Synthetic proboe
  source 1..50: mol_type = other DNA
atctcacctaacatctatttcctatttatatttttcacataatattttac

SEQ ID NO: 8 - DNA, 50 bp, synthetic construct
  misc_feature 1..50: note = Synthetic proboe
  source 1..50: mol_type = other DNA
ctaaaaattccaaaccaaaaaaaaactcctaacacatattaaaaaaatca

SEQ ID NO: 9 - DNA, 50 bp, synthetic construct
  misc_feature 1..50: note = Synthetic proboe
  source 1..50: mol_type = other DNA
aaaatcctaacrctacttaaaaccaacaactatcaattaatcraaaactc

(19) United States
(12) Patent Application Publication
(10) Pub. No.: US 2023/0287501 A1
(43) Pub. Date: Sep. 14, 2023

(54) IDENTIFICATION AND TREATMENT OF OPIOID USE DISORDER

(71) Applicant: BATTELLE MEMORIAL INSTITUTE, Columbus, OH (US)

(72) Inventors: Rachel R. SPURBECK, Columbus, OH (US); Jared SHUETTER, Columbus, OH (US); Brandon HILL, Columbus, OH (US); Angela MINARD-SMITH, Marysville, OH (US); Lydia STEWART, Jessup, MD (US)

(21) Appl. No.: 18/065,307

(22) Filed: Dec. 13, 2022

Related U.S. Application Data
(60) Provisional application No. 63/289,340, filed on Dec. 14, 2021.

(51) Int. Cl. C12Q 1/6883; A61K 48/00

(52) U.S. Cl. CPC C12Q 1/6883; A61K 48/0066; C12Q 2600/106; C12Q 2600/118; C12Q 2600/154; C12Q 2600/156

(57) ABSTRACT

Compositions and methods are provided for identifying individuals at risk for developing OUD.

### CROSS REFERENCE TO RELATED APPLICATIONS

This application claims priority to U.S. Provisional Pat. Application No. 63/289,340 filed on Dec. 14, 2021, the disclosure of which is expressly incorporated herein.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 13 kilobytes xml file named “920006-375584.xml,” created on Dec. 12, 2022.

### BACKGROUND

Although the opioid epidemic was declared a nationwide Public Health Emergency on Oct. 27, 2017, opioid use and overdoses have continued to increase. In 2020 over 92,000 Americans died from opioid overdoses, which is approximately a 30% increase over 2019. Opioid Use Disorder (OUD) is a substance use disorder characterized by a problematic pattern of opioid use leading to problems or distress. Unique to OUD, there is a strong, rapid onset to physical dependence (4-8 weeks) and, in chronic users, violent withdrawal side effects when use is abruptly stopped. The development of OUD is due to a mixture of environmental and genetic factors, which need to be better characterized to enable effective screening of patients prior to and during opioid prescription use.

Between 8-12 percent of people who take prescription pain medications develop OUD, therefore, screening patients prior to and during an opioid prescription could reduce OUD prevalence. Currently, tools assess risk of OUD through self-reported questionnaires such as Opioid Risk Tool and the Screener and Opioid Assessment for Patients with Pain (SOAPP®), which calculate risk based on the patient’s environment, medical history, and family history. These tools rely on a subject’s memory, which can be biased or inaccurate. Furthermore, the SOAPP-Revised was shown to only identify 52% of aberrant drug-related behavior with a sensitivity of 80%, indicating that OUD diagnostic tools have significant room for improvement.

Genetic factors have been identified that account for approximately half of the individuals that have an increased risk of developing addiction. Accordingly, molecular tests could improve diagnosis of patients predisposed to have OUD prior to treatment, however, genetics does not account for all the risk. Therefore, a more predictive multivariant clinical risk scoring approach that removes the subjectivity of completely questionnaire-based tools and increases the precision of current genetics tools is needed.

The epigenome is a complex of molecular mechanisms that can alter gene expression based on environmental signals by modifying adducts (methylation, histone modifications, open/closed chromatin) on deoxyribonucleic acid (DNA). Changes in gene expression leads to changes in the proteins produced by the body, which is why the epigenome is important to monitor for changes associated with OUD. Several studies have indicated a role for methylation or histone modifications in substance use disorders like OUD. By combining epigenetics and static genetic markers, more OUD risk factors will be accounted for while removing the subjectivity of tools relying on human memory.

As disclosed herein molecular based diagnostics and prognostics tools are provided that combine static genetic mutational analysis with environment-responsive molecular targets, the epigenome, to account for both genetic and environmental factors that influence OUD to enable more accurate risk assessments that can be used to improve OUD prevention and management strategies.

### SUMMARY

Twenty percent of people who are prescribed an opioid become addicted and have Opioid Use Disorder (OUD). This leads to billions of dollars being spent to treat and manage opioid addiction, as well as several preventable deaths due to overdose. The diagnostic tools disclosed herein can be used prior to prescription of opioids, and allow clinicians to pick the best course of treatment for their patients to avoid OUD. This will reduce the economic burden of OUD and unnecessary deaths due to overdoses.

In accordance with one embodiment epigenetic marks are compared between people with OUD and those who do not have OUD to identify a baseline epigenetic state that can be used to diagnose individuals at risk of OUD. Furthermore, as the methylome is responsive to the environment and can change with time, continued monitoring the epigenetic state of the patient can detect changes that indicate an increased risk of the patient developing OUD. Thus monitoring the methylome will enable clinicians to modify treatment regimens as needed to reduce the risk of OUD in their patients who may have been prescribed an opioid.

In accordance with one embodiment a method of detecting a set of molecular biomarkers in the DNA of patients that are associated with Opioid Use Disorder (OUD) is provided. In one embodiment the method comprises the steps of recovering a biological sample from the patient, recovering DNA from the biological sample, and detecting the methylated state of 1, 2, 3, 4, 5, 6 or 7 genes selected from the group consisting of HOXA5, HOX6A, BLCAP, NNAT, RAI14, ZNF710, IL17B, RASSF4, ZMIZ1, ZNHIT6 and CDKAL1, or any of the intergenic regions identified in Table 1. In one embodiment the method comprises the steps of recovering a biological sample from the patient, recovering DNA from the biological sample, and detecting the methylated state of 1, 2, 3, 4, 5, 6 or 7 sites in genes selected from the group consisting of HOXA5, BLCAP, CDKAL1. In one embodiment the method comprises the steps of recovering a biological sample from the patient, recovering DNA from the biological sample, and detecting the methylated state of 1, 2, 3, 4, 5, 6 or 7 sites in genes selected from the group consisting of HOXA5, BLCAP, RAI14, ZNF710, IL17B, RASSF4, ZMIZ1, ZNHIT6 and CDKAL1 or in any of the intergenic regions identified by DMR2 probes 15 (cg11383134; SEQ ID NO: 8) and 20 (cg19636627 SEQ ID NO: 9). In one embodiment the methylated state of each of the RAI14, ZNF710, IL17B, RASSF4, ZMIZ1, ZNHIT6 and CDKAL1 genes is determined by contacting a DNA sample from the patient with a probe selected from the group of cg05666482 (SEQ ID NO: 1), cg16700210 (SEQ ID NO: 2), cg02707345 (SEQ ID NO: 3), cg15673864 (SEQ ID NO: 4), cg16112766 (SEQ ID NO: 5), cg06355651 (SEQ ID NO: 6) and cg08559711 (SEQ ID NO: 7) or complements thereof. The biological sample recovered from the patient can be any tissue or bodily fluid that is known to contain DNA. In one embodiment the biological sample is blood or a blood component. In one embodiment the biological sample comprises cells recovered from a cheek/buccal swabs.

Furthermore, as the methylome is responsive to the environment and can change during development of OUD, monitoring the methylome will enable clinicians to modify treatment regimens as needed to reduce OUD in their patients who may have been prescribed an opioid. In accordance with one embodiment the diagnostic procedures disclosed herein are used to analyze the methylome, including continued monitoring of the methylome during administration of opioid therapy to reduce the number of people suffering from OUD, reduce the number of overdoses, and help mitigate the opioid epidemic in the USA. By monitoring the methylome over time while an individual is on an opioid, a determination can be made to reduce or eliminate administration of an opioid, or switch from an opioid therapeutic to a different pain treatment to avoid OUD.

In addition to monitoring the methylome, to assess OUD risk in patients, the analysis can be supplemented by an analysis of static genetic markers such as single nucleotide polymorphisms. In accordance with one embodiment the following 25 single nucleotide polymorphisms have been identified as having diagnostic potential for OUD and OUD risk: GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846 DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131. By targeting the 25 single nucleotide polymorphisms listed, either all together, individually, or in any combination of 1-25 of these single nucleotide polymorphisms, optionally in conjunction with an analysis of the patient’s methylome, the genetic risk of OUD will be determined for individuals prior to prescriptions. This will reduce the number of people who get opioid use disorder after taking a prescription, and enable continued use of opioids for pain management in the subset of the population that are not susceptible to OUD.

Opioid Use Disorder (OUD) occurs due to both genetic and environmental factors. Accordingly, in one embodiment an effective strategy for diagnosing and monitoring for OUD, includes screening for both static genetic polymorphisms that cause or are markers of OUD as well as environmentally influenced changes, which can be monitored through the epigenome. Therefore, a molecular assay that covers a panel of single nucleotide polymorphisms (SNPs) and methylated sites in the genome could encompass all the risk potential for OUD, providing higher precision and accuracy than either SNPs or methylation alone. Therefore, in one embodiment a method of diagnosing a subject as one at risk for developing OUD is provided wherein the method comprises conducting a molecular assay on a DNA sample recovered from the subject that screens for a panel of single nucleotide polymorphisms (SNPs) and methylated sites in the genome that are associated with OUD. In one embodiment, the panel of single nucleotide polymorphisms (SNPs) used in the method of the present disclosure comprises two or more SNPS selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131, optionally wherein the panel comprises the SNP markers GRIN3A (rs17189632), RGS9-2 (rs1530351), COMT (rs4680), CNIH3 (rs1436175) and DRD2 (rs4436578). In one embodiment, the panel of single nucleotide polymorphisms (SNPs) used in the method of the present disclosure comprises two or more SNPS selected from the group consisting of GRIN3A rs17189632, RGS9-2 rs1530351, CNIH3 rs1436175, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, BDNFOS/antiBDNF rs988712, DRD2 rs2283265, and DRD2 rs1076560. Furthermore, in one embodiment, the panel of methylated sites used in the method of the present disclosure comprises two or more methylated sites selected from the group consisting of HOXA5, HOX6A, BLCAP, NNAT, RAI14, ZNF710, IL17B, RASSF4, ZMIZ1, ZNHIT6 and CDKAL1.

In accordance with one embodiment a method of using opioids for treating pain in subjects is provided while diminishing the risk for developing Opioid Use Disorder (OUD). In one embodiment the method comprises identifying a subject being at risk of OUD by detecting the methylated state of genes selected from the group consisting of HOXA5, HOX6A, BLCAP, NNAT, RAI14, ZNF710, IL17B, RASSF4, ZMIZ1, ZNHIT6 and CDKAL1 or any of the intergenic regions identified in Table 1; and identifying individuals having an altered methylation of at least two of said HOXA5, HOX6A, BLCAP, NNAT, RAI14, ZNF710, IL17B, RASSF4, ZMIZ1, ZNHIT6 and CDKAL1 genes or intergenic regions identified in Table 1, relative to a control. Those individuals identified as having, or at risk of developing OUD, are then treated by either avoiding opioid administration altogether, or switching to a different pain treatment to avoid OUD when an OUD methylation pattern is detected. In one embodiment the individuals having an increased methylation of at least 2, 3, 4, 5, 6 or 7 of said HOXA5, HOX6A, BLCAP, NNAT, RAI14, ZNF710, IL17B, RASSF4, ZMIZ1, ZNHIT6 and CDKAL1 genes intergenic regions identified in Table 1 relative to a control are identified as individuals either having, or risk for developing, Opioid Use Disorder (OUD) upon further exposure to opioids. In one embodiment the method further comprises analyzing one or more static genetic markers of OUD, including for example, detecting the presence of SNP markers in a DNA sample from the subject. In accordance with one embodiment a diagnosis of OUD is associated with differential methylation of the HOXA5, HOX6A, BLCAP and NNAT genes, and the intergenic region between MOG and pseudogene SUMO2P1, optionally in combination with the detection of one or more static genetic polymorphisms (e.g. SNPS).

In one embodiment SNP markers used for identification of subjects at risk of OUD are selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131. In one embodiment the SNP marker used for identification of subjects at risk of OUD is selected from the group consisting of GRIN3A rs17189632, RGS9-2 rs1530351, CNIH3 rs1436175, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, BDNFOS/antiBDNF rs988712, DRD2 rs2283265, and DRD2 rs1076560. In one embodiment the SNP marker used for identification of subjects at risk of OUD is selected from the group consisting of CNIH3 rs1436175, OPRD1 rs678849, DRD3 rs324029, GRIN3A rs17189632, CYP2C19 rs4244285, DCC rs12607853, CYP1A2 rs762551, CYP3A5 rs15524, CYP3A5 rs776746, RGS9-2 rs1530351, ABCB1/ MDR1 rs1045642, TAOK3 rs795484, FAAH rs324420, and OPRD1 rs508448.

In one embodiment a method of screening for subjects at risk of OUD includes detecting the static makers of CNIH3 rs1436175, OPRD1 rs678849, DRD3 rs324029, GRIN3A rs17189632, CYP2C19 rs4244285, DCC rs12607853, CYP1A2 rs762551, CYP3A5 rs15524, RGS9-2 rs1530351, and TAOK3 rs795484, in combination with two differential methylation markers, optionally wherein the methylated sites are present in genes selected from the group consisting of RAI14, ZNF710, IL17B, RASSF4, ZMIZ1, ZNHIT6 and CDKAL1, optionally wherein the methylation markers are detected with DMR2 probes 15 (cg11383134 0.7045 0.9040 0.1995 0.2883 chr6:29648590 IGR) and 20 (cg19636627 0.6307 0.8703 0.2396 0.3222 chr6:29649084 IGR).

The differentially methylate DNA sequences and SNPs identified herein as being associated with OUD can be used individually or in any combination as the targets in a diagnostic or prognostic molecular test using techniques known to those skilled in the art. In accordance with one embodiment, the molecular test used for detecting the OUD associated SNPs or methylation markers in DNA isolated from a patient’s biological sample is selected from the group of tests consisting of one: microarray technology, bead array, qPCR, or sequencing assay. In on embodiment the microarray, bead array, or sequencing assay is designed to target the specific sites identified herein or queried through bioinformatic analysis of whole genome arrays or sequencing assays. Quantitative PCR will be specific to the targets identified in in the present disclosure. The general methods for each of these tests are public knowledge..

In accordance with one embodiment a method of treating a subject having OUD is provided wherein the method comprises introducing an epieffector into the cells of said subject, wherein said epieffector comprises an epigenetic regulator fused to a sequence specific endonuclease, optionally a dCas9 or zinc finger protein, to target removal of methylation sites present in a gene selected from the group consisting of RAI14, ZNF710, IL17B, RASSF4, ZMIZ1, ZNHIT6 and CDKAL1. In one embodiment the epigenetic regulator is an enzyme or enzyme complex that selectively removes methyl groups (or alters the methyl group resulting in subsequent removal of the methyl group) from targeted sites in the of RAI14, ZNF710, IL17B, RASSF4, ZMIZ1, ZNHIT6 and CDKAL1 genes. In one embodiment the epigenetic regulator is a ten-eleven translocation (TET) enzyme. Ten-eleven translocation (TET) enzymes participate in the initial step of DNA demethylation by oxidizing 5mC to 5hmC, which can lead to demethylation. In one embodiment the epieffector is encapsulated or bound to a delivery system and introduced into the cells of said subject.

### BRIEF DESCRIPTION OF THE DRAWINGS

FIG. 1: is a graph of the average normalized methylation of seven differentially methylated probes were identified by EPIC array analysis, showing the difference between control and OUD methylation. Probes cg05666482, cg16700210, and cg02707345 are from genes RAI14, ZNF710, and IL17B respectively; probes cg15673864, cg16112766, and cg06355651 are from genes RASSF4, ZMIZ1, and ZNHIT6 respectively; and probe cg08559711 is from gene CDKAL1.

FIG. 2 Eight probes were identified by the LASSO model to distinguish OUD from controls. Probe cg08559711 is from gene CDKAL1; probes cg14014955, cg14013695, cg16997642, cg23204968 and cg19643053 are from the gene HOXA5; probe cg20961245 is from an intergenic region (IGR); and probe cg18433380 is from the gene BLCAP.

FIG. 3 is a graph showing the 29 probes identified by the Random Forest model as distinguishing features between OUD and controls. Probes cg05666482, cg16700210, and cg02707345 are from genes RAI14, ZNF710, and IL17B respectively; probes cg15673864, cg16112766, and cg06355651 are from genes RASSF4, ZMIZ1, and ZNHIT6 respectively; probe cg08559711 is from gene CDKAL1; probes cg12015737, cg07049592, cg08070327, cg13694927, cg00969405, cg17432857, cg26023912, cg01323381, cg16997642, cg14014955 are from the gene HOXA5; cg21641458, cg24040595 are from the gene HOXA6; probe cg11174847 is from gene NNAT; probes cg15708526, cg12644888, and cg08041448 are from an intergenic region (IGR); and probes cg22510412, cg26083330, cg22551578, cg22421148, cg22298088, cg07156273 are from the gene BLCAP.

FIG. 4. presents a LASSO model utilizing 13 SNPs to differentiate OUD from controls.

FIG. 5 presents a random forest model utilizing all 25 SNPs to differentiate OUD from controls.

FIG. 6 presents a LASSO model using a mix of methylation probes (cg19636627 and cg11383134 from intergenic regions) and SNPs (10) to differentiate OUD from controls.

FIG. 7 presents a Random forest model using a mix of methylation probes (56) and SNPs (3) to differentiate OUD from controls.

### DEFINITIONS

In describing and claiming the methods, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein the terms “effective amount” or “therapeutically effective amount” of a compound refers to a nontoxic but sufficient amount of the compound to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein the term “subject” means an animal including but not limited to, humans, domesticated animals including horses, dogs, cats, cattle, and the like, rodents, reptiles, and amphibians.

As used herein the term “patient” without further designation is intended to encompass any warm blooded vertebrate domesticated animal (including for example, but not limited to livestock, horses, cats, dogs and other pets) and humans receiving a therapeutic treatment whether or not under the supervision of a physician.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviation of the symptoms associated with a specific disorder or condition and/or preventing or eliminating said symptoms.

### EMBODIMENTS

As disclosed herein compositions and methods are provided for identifying subjects that are at enhanced risk of developing OUD upon administration of opioids. More particularly, in one embodiment epigenetic marks are identified that can be used to diagnose, predict, and monitor patients for OUD to identify people who should not be given opioids, and monitor those who are on opioids to prevent more people getting OUD. As disclosed herein applicant has identified sets of epigenetic and static genetic markers that are diagnostic for individuals susceptible to developing OUD upon administration, or continued administration, of opioids.

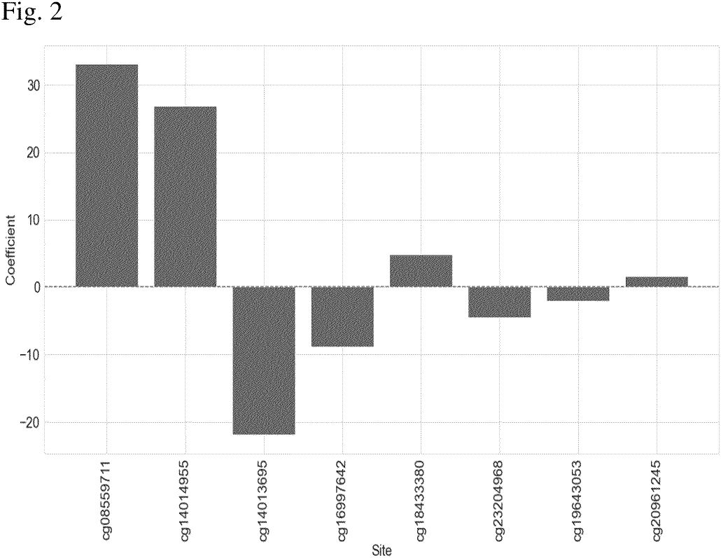

In accordance with one embodiment a patient is screened for risk factors prior to the administration of an opioid. In one embodiment, a subject is identified as being at risk of OUD by detecting the methylated state of one or more genes selected from the group consisting of HOXA5, HOX6A, BLCAP, NNAT, RAI14, ZNF710, IL17B, RASSF4, ZMIZ1, ZNHIT6 and CDKAL1, and determining if 1, 2, 3, 4, 5, 6, 7, 8 9, 10, or all 11 genes have an altered methylation pattern (e.g., elevated methylation) relative to a control. In one embodiment, a subject is identified as being at risk of OUD by detecting the methylated state of one or more intergenic regions including for example an IGR covered by probe cg27151303 at chromosome 7, nt position 27184821 and probe cg05579037 which is at chromosome 7 nucleotide position 27184853, and another IGR covered by 21 probes from chromosome 6 nucleotide position 29648161 thru 29649092. In total, 103 probes were found to be differentially methylated, seven individually were able to differentiate OUD and non-OUD (See FIG. 2), and 96 were able to differentiate OUD from non-OUD when associated into three regions (See Table 1).

TABLE 1

List of differentially methylated probes identified by EPIC array analysis

| | |
|---|---|
| DMR1 | |
| cg23936031 0.5586 0.6389 0.0803 0.3670 chr7:27183133 HOXA5 1stExon | DMR probe 1 |
| cg09549073 0.4930 0.6195 0.1265 0.2626 chr7:27183274 HOXA5 5′UTR | DMR probe 2 |
| cg04863892 0.4917 0.6088 0.1171 0.4086 chr7:27183375 HOXA5 TSS200 | DMR probe 3 |
| cg09207400 0.4503 0.5524 0.1022 0.2748 chr7:27183382 HOXA5 TSS200 | DMR probe 4 |
| cg19759481 0.4994 0.6422 0.1429 0.3258 chr7:27183401 HOXA5 TSS200 | DMR probe 5 |
| cg02916332 0.5602 0.6713 0.1110 0.3399 chr7:27183591 HOXA5 TSS 1500 | DMR probe 6 |
| cg17569124 0.4346 0.5682 0.1336 0.2633 chr7:27183643 HOXA5 TSS 1500 | DMR probe 7 |
| cg02005600 0.5659 0.7197 0.1539 0.2335 chr7:27183686 HOXA5 TSS1500 | DMR probe 8 |
| cg25307665 0.4715 0.5953 0.1238 0.1903 chr7:27183694 HOXA5 TSS1500 | DMR probe 9 |
| cg14014955 0.5608 0.7489 0.1881 0.1384 chr7:27183701 HOXA5 TSS1500 | DMR probe 10 |
| cg02646423 0.5414 0.6832 0.1418 0.3335 chr7:27183794 HOXA5 TSS1500 | DMR probe 11 |
| cg20517050 0.5797 0.7842 0.2044 0.1850 chr7:27183806 HOXA5 TSS 1500 | DMR probe 12 |
| cg23204968 0.6883 0.7960 0.1077 0.4380 chr7:27183816 HOXA5 TSS1500 | DMR probe 13 |
| cg14058329 0.7772 0.8471 0.0699 0.2309 chr7:27183946 HOXA5 TSS1500 | DMR probe 14 |
| cg03207666 0.8194 0.8995 0.0801 0.1819 chr7:27183950 HOXA5 TSS1500 | DMR probe 15 |
| cg23454797 0.7353 0.8495 0.1143 0.1722 chr7:27183990 HOXA5 TSS1500 | DMR probe 16 |
| cg12015737 0.8559 0.9103 0.0543 0.1771 chr7:27184030 HOXA5 TSS1500 | DMR probe 17 |
| cg08070327 0.8262 0.9150 0.0888 0.2773 chr7:27184059 HOXA5 TSS1500 | DMR probe 18 |
| cg25506432 0.6437 0.7458 0.1021 0.2642 chr7:27184065 HOXA5 TSS1500 | DMR probe 19 |
| cg16997642 0.8791 0.9425 0.0634 0.3288 chr7:27184159 HOXA5 TSS1500 | DMR probe 20 |
| cg20817131 0.6785 0.7905 0.1120 0.3461 chr7:27184167 HOXA5 TSS1500 | DMR probe 21 |
| cg14013695 0.8110 0.8662 0.0553 0.2917 chr7:27184176 HOXA5 TSS1500 | DMR probe 22 |
| cg25390165 0.6500 0.7794 0.1294 0.1003 chr7:27184188 HOXA5 TSS1500 | DMR probe 23 |
| cg01323381 0.6285 0.7635 0.1351 0.1706 chr7:27184264 HOXA5 TSS1500 | DMR probe 24 |
| cg19643053 0.8291 0.8798 0.0507 0.3432 chr7:27184271 HOXA5 TSS1500 | DMR probe 25 |
| cg26023912 0.5483 0.6794 0.1311 0.1796 chr7:27184369 HOXA5 TSS1500 | DMR probe 26 |
| cg14882265 0.5915 0.7208 0.1292 0.1565 chr7:27184375 HOXA5 TSS1500 | DMR probe 27 |
| cg17432857 0.5323 0.6749 0.1426 0.2313 chr7:27184438 HOXA5 TSS1500 | DMR probe 28 |
| cg00969405 0.6276 0.7740 0.1465 0.1954 chr7:27184441 HOXA5 TSS1500 | DMR probe 29 |
| cg07049592 0.6471 0.7609 0.1138 0.0960 chr7:27184450 HOXA5 TSS1500 | DMR probe 30 |
| cg02106682 0.5563 0.6496 0.0933 0.2781 chr7:27184461 HOXA5 TSS1500 | DMR probe 31 |
| cg03368099 0.5093 0.5861 0.0768 0.3261 chr7:27184521 HOXA5 TSS1500 | DMR probe 32 |
| cg01748892 0.3955 0.4306 0.0351 0.3559 chr7:27184667 HOXA5 TSS1500 | DMR probe 33 |
| cg13694927 0.4097 0.4430 0.0333 0.5209 chr7:27184712 HOXA5 TSS1500 | DMR probe 34 |
| cg03744763 0.2578 0.3334 0.0756 0.3047 chr7:27184737 HOXA5 TSS1500 | DMR probe 35 |
| cg27151303 0.4228 0.5178 0.0950 0.2815 chr7:27184821 IGR | DMR probe 36 |
| cg05579037 0.6551 0.7573 0.1022 0.3130 chr7:27184853 IGR | DMR probe 37 |
| cg21641458 0.6788 0.7873 0.1086 0.1279 chr7:27185136 HOXA6 3′UTR | DMR probe 38 |
| cg09343092 0.6828 0.7596 0.0768 0.1105 chr7:27185282 HOXA6 Body | DMR probe 39 |
| cg24040595 0.7475 0.8616 0.1141 0.1180 chr7:27185512 HOXA6 Body | DMR probe 40 |
| cg06465806 0.7614 0.8556 0.0942 0.2314 chr7:27185732 HOXA6 Body | DMR probe 41 |
| DMR2 | |
| cg25978138 0.8584 0.9587 0.1003 0.3446 chr6:29648161 IGR | DMR 2 probe 1 |
| cg11747594 0.6948 0.8796 0.1848 0.3476 chr6:29648225 IGR | DMR 2 probe 2 |
| cg15708526 0.7690 0.8892 0.1202 0.3690 chr6:29648271 IGR | DMR 2 probe 3 |
| cg04071440 0.7127 0.8467 0.1340 0.2925 chr6:29648275 IGR | DMR 2 probe 4 |
| cg08022281 0.5044 0.6005 0.0961 0.2811 chr6:29648345 IGR | DMR 2 probe 5 |
| cg10648573 0.5321 0.6418 0.1097 0.2382 chr6:29648348 IGR | DMR 2 probe 6 |
| cg12644888 0.5410 0.6524 0.1114 0.3341 chr6:29648360 IGR | DMR 2 probe 7 |
| cg22494932 0.7750 0.9358 0.1608 0.3603 chr6:29648379 IGR | DMR 2 probe 8 |
| cg25699073 0.7878 0.9349 0.1470 0.3284 chr6:29648381 IGR | DMR 2 probe 9 |
| cg07134666 0.7217 0.8978 0.1761 0.4094 chr6:29648400 IGR | DMR 2 probe 10 |
| cg00588198 0.5799 0.8093 0.2293 0.2409 chr6:29648452 IGR | DMR 2 probe 11 |
| cg06032337 0.2126 0.3516 0.1390 0.6803 chr6:29648468 IGR | DMR 2 probe 12 |
| cg16885113 0.7445 0.9158 0.1714 0.3883 chr6:29648507 IGR | DMR 2 probe 13 |
| cg20228636 0.6660 0.8749 0.2089 0.2633 chr6:29648525 IGR | DMR 2 probe 14 |
| cg11383134 0.7045 0.9040 0.1995 0.2883 chr6:29648590 IGR | DMR 2 probe 15 |
| cg03449857 0.6872 0.8722 0.1850 0.4138 chr6:29648623 IGR | DMR 2 probe 16 |
| cg15570656 0.6795 0.8814 0.2019 0.3920 chr6:29648628 IGR | DMR 2 probe 17 |
| cg08041448 0.4681 0.6336 0.1655 0.4815 chr6:29648901 IGR | DMR 2 probe 18 |
| cg24100841 0.5500 0.7810 0.2310 0.3469 chr6:29649024 IGR | DMR 2 probe 19 |
| cg19636627 0.6307 0.8703 0.2396 0.3222 chr6:29649084 IGR | DMR 2 probe 20 |
| cg20961245 0.4563 0.7272 0.2710 0.5029 chr6:29649092 IGR | DMR 2 probe 21 |
| DMR3 | |
| cg23757721 0.6294 0.7273 0.0979 0.2313 chr20:36148604 BLCAP 5′UTR | DMR 3 probe 1 |
| cg26083330 0.6731 0.7296 0.0565 0.2890 chr20:36148615 BLCAP 5′UTR | DMR 3 probe 2 |
| cg22551578 0.5151 0.5919 0.0768 0.2936 chr20:36148620 BLCAP 5′UTR | DMR 3 probe 3 |
| cg04489586 0.6168 0.6698 0.0531 0.2584 chr20:36148642 BLCAP 5′UTR | DMR 3 probe 4 |
| cg23605670 0.5467 0.5981 0.0513 0.4245 chr20:36148672 BLCAP 5′UTR | DMR 3 probe 5 |
| cg12862537 0.5130 0.6016 0.0886 0.3497 chr20:36148679 BLCAP 5′UTR | DMR 3 probe 6 |
| cg10981598 0.5570 0.6047 0.0477 0.4977 chr20:36148699 BLCAP 5′UTR | DMR 3 probe 7 |
| cg13790727 0.6835 0.7392 0.0557 0.3080 chr20:36148738 BLCAP 5′UTR | DMR 3 probe 8 |
| cg20783699 0.6612 0.7164 0.0551 0.4381 chr20:36148767 BLCAP 5′UTR | DMR 3 probe 9 |
| cg25712981 0.6974 0.7470 0.0496 0.4130 chr20:36148775 BLCAP 5′UTR | DMR 3 probe 10 |
| cg03615235 0.6898 0.7495 0.0597 0.3770 chr20:36148779 BLCAP 5′UTR | DMR 3 probe 11 |
| cg17643025 0.7172 0.7786 0.0614 0.4134 chr20:36148791 BLCAP 5′UTR | DMR 3 probe 12 |
| cg24762053 0.6544 0.7251 0.0707 0.3763 chr20:36148798 BLCAP 5′UTR | DMR 3 probe 13 |
| cg14469070 0.6982 0.7705 0.0723 0.4211 chr20:36148803 BLCAP 5′UTR | DMR 3 probe 14 |
| cg22298088 0.6240 0.7220 0.0980 0.3332 chr20:36148860 BLCAP 5′UTR | DMR 3 probe 15 |
| cg22943498 0.6324 0.7115 0.0791 0.3525 chr20:36148928 BLCAP 5′UTR | DMR 3 probe 16 |
| cg14765818 0.5641 0.6214 0.0573 0.2663 chr20:36148954 BLCAP 5′UTR | DMR 3 probe 17 |
| cg15473473 0.5484 0.6579 0.1094 0.2683 chr20:36148994 BLCAP 5′UTR | DMR 3 probe 18 |
| cg07156273 0.5809 0.6817 0.1009 0.2991 chr20:36149013 BLCAP 5′UTR | DMR 3 probe 19 |
| cg01466133 0.5916 0.6947 0.1031 0.3133 chr20:36149022 BLCAP 5′UTR | DMR 3 probe 20 |
| cg24675557 0.5913 0.6732 0.0819 0.2959 chr20:36149081 BLCAP 5′UTR | DMR 3 probe 21 |
| cg20479660 0.5547 0.6514 0.0968 0.2987 chr20:36149112 BLCAP 5′UTR | DMR 3 probe 22 |
| cg22421148 0.5850 0.6800 0.0949 0.2380 chr20:36149119 BLCAP 5′UTR | DMR 3 probe 23 |
| cg22510412 0.6125 0.7335 0.1209 0.2462 chr20:36149121 BLCAP 5′UTR | DMR 3 probe 24 |
| cg03061677 0.5606 0.6506 0.0899 0.2655 chr20:36149185 BLCAP 5′UTR | DMR 3 probe 25 |
| cg00576435 0.5449 0.6382 0.0933 0.2519 chr20:36149188 BLCAP 5′UTR | DMR 3 probe 26 |
| cg18433380 0.5266 0.6414 0.1148 0.2482 chr20:36149194 BLCAP 5′UTR | DMR 3 probe 27 |
| cg16648571 0.5015 0.5677 0.0661 0.2840 chr20:36149231 BLCAP 5′UTR | DMR 3 probe 28 |
| cg04810287 0.5262 0.6342 0.1080 0.3253 chr20:36149271 BLCAP 5′UTR | DMR 3 probe 29 |
| cg25962605 0.4809 0.5538 0.0728 0.2745 chr20:36149452 BLCAP 5′UTR | DMR 3 probe 30 |
| cg21588305 0.5835 0.6805 0.0971 0.2154 chr20:36149455 BLCAP 5′UTR | DMR 3 probe 31 |
| cg13708635 0.4964 0.5270 0.0306 0.4438 chr20:36149656 NNAT 5′UTR | DMR 3 probe 32 |
| cg11174847 0.5316 0.5784 0.0468 0.2223 chr20:36149706 NNAT 5′UTR | DMR 3 probe 33 |
| cg23566503 0.5068 0.5612 0.0545 0.2711 chr20:36149750 NNAT 1stExon | DMR 3 probe 34 |

In one embodiment the control represents the level of methylation of the listed genes, or intergenic regions, based on average population data. In one embodiment the control is based on the level of methylation of the respective genes, or intergenic regions, based on detected levels in individuals not susceptible to OUD. In one embodiment, a subject is identified as being at risk of OUD by detecting the methylated state of one or more genes selected from the group consisting of RAI14, ZNF710, IL17B, RASSF4, ZMIZ1, ZNHIT6 and CDKAL1; and determining if 1, 2, 3, 4, 5, 6, or all 7 genes have increased methylation relative to a control, wherein the detection of elevated methylation rates in the referenced genes is diagnostic for enhanced risk of OUD. In one embodiment the method is based on the detection of 5N methylcytosine (5mC), optionally the detection of a C-methylated dipeptide of CpG, optionally in one or more loci of one or more genes selected from the group of HOXA5, HOX6A, BLCAP, NNAT, RAI14, ZNF710, IL17B, RASSF4, ZMIZ1, ZNHIT6 and CDKAL1, or any of the intergenic regions identified in Table 1. In one embodiment, a subject is identified as being at risk of OUD by detecting methylation at seven difference loci of seven genes using probes cg05666482, cg16700210, and cg02707345 from genes RAI14, ZNF710, and IL17B respectively; probes cg15673864, cg16112766, and cg06355651 from genes RASSF4, ZMIZ1, and ZNHIT6 respectively; and probe cg08559711 from gene CDKAL1. In one embodiment, a subject is identified as being at risk of OUD by detecting methylation at seven difference loci of three genes (CDKAL1, HOXA5 and BLCAP) optionally using probe using probe cg08559711 from gene CDKAL1; probes cg14014955, cg14013695, cg16997642, cg23204968 and cg19643053 from the gene HOXA5; probe cg20961245 from an intergenic region (IGR); and probe cg18433380 from the gene BLCAP.

In accordance with one embodiment a patient receiving an opioid is monitored throughout the therapeutic administration of an opioid to identify any epigenetic alterations that indicate an increased risk for developing OUD. In one embodiment, the steps of monitoring a patient receiving an opioid comprises screening a DNA sample recovered from the patient to detect alterations in the methylated state of one or more genes selected from the group consisting of HOXA5, HOX6A, BLCAP, NNAT, RAI14, ZNF710, IL17B, RASSF4, ZMIZ1, ZNHIT6 and CDKAL1, or any of the intergenic regions identified in Table 1. In one embodiment, a subject is monitored to detect alterations in the methylated state of one or more genes selected from RAI14, ZNF710, RASSF4, ZMIZ1, ZNHIT6, CDKAL1 and IL17B, optionally wherein seven difference loci of seven genes are monitored using probes cg05666482, cg16700210, and cg02707345 from genes RAI14, ZNF710, and IL17B respectively; probes cg15673864, cg16112766, and cg06355651 from genes RASSF4, ZMIZ1, and ZNHIT6 respectively; and probe cg08559711 from gene CDKAL1. In one embodiment, a subject is monitored to detect alterations in the methylated state of one or more genes selected from seven difference loci of three genes (CDKAL1, HOXA5 and BLCAP) optionally using probe cg08559711 from gene CDKAL1; probes cg14014955, cg14013695, cg16997642, cg23204968 and cg19643053 from the gene HOXA5; probe cg20961245 from an intergenic region (IGR); and probe cg18433380 from the gene BLCAP.

In one embodiment the methylated state of one or more genes selected from the group consisting of RAI14, ZNF710, IL17B, RASSF4, ZMIZ1, ZNHIT6 and CDKAL1 is monitored relative to the methylated state of those genes prior to receiving an opioid treatment, or relative to a control. In one embodiment the control represents the level of methylation of the listed genes based on average population data. In one embodiment the control is based on the level of methylation of the respective genes based on detected level in individuals not susceptible to OUD. In one embodiment, a subject is identified as being at risk of OUD if during the course of administering an opioid the methylated state of one or more genes selected from the group consisting of HOXA5, HOX6A, BLCAP, NNAT, RAI14, ZNF710, IL17B, RASSF4, ZMIZ1, ZNHIT6 and CDKAL1, or any of the intergenic regions identified in Table 1. ; is increased relative to a control. In one embodiment the method is based on the detection of 5N methylcytosine (5mC), optionally the detection of a C-methylated dipeptide of CpG.

In one embodiment, a subject is identified as being at risk of OUD by detecting the methylated state of one or more genes in conjunction with detecting the presence of certain static molecular markers associated with OUD. In this embodiment the methylated state of one or more genes selected from those identified in Table 1, optionally one or more genes selected from the group consisting of HOXA5, HOX6A, BLCAP, NNAT, RAI14, ZNF710, IL17B, RASSF4, ZMIZ1, ZNHIT6 and CDKAL1, or any of the intergenic regions identified in Table 1 is determined, and DNA from a biological sample of the patient (e.g., tissue sample, blood or a blood component) is screen for the presence of one or more SNPs selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131, wherein elevated methylation levels in of RAI14, ZNF710, IL17B, RASSF4, ZMIZ1, ZNHIT6 and CDKAL1 in conjunction with the detection of one or more of the above SNPs identifies patients at risk of developing OUD.

In one embodiment a patient is identified as being at risk of developing OUD, if two or more genes selected from the group consisting of RAI14, ZNF710, IL17B, RASSF4, ZMIZ1, ZNHIT6 and CDKAL1 have elevated methylation relative to a control and two or more SNPs selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131 are detected in the patient’s biological sample. In one embodiment the control represents the level of methylation of the listed genes based on average population data. In one embodiment the control is based on the level of methylation of the respective genes based on detected level in individuals not susceptible to OUD.

In one embodiment a patient is identified as being at risk of developing OUD, based on the identification of a mix of methylation probes (cg19636627 and cg11383134 from intergenic regions) and identification of SNPs CNIH3 rs1436175, DRD3 rs324029, OPRD1 rs678849, CYP3A5 rs15524, GRIN3A rs17189632, CYP2C19 rs4244285, DCC rs12607853, CYP1A2 rs762551, TAOK3 rs795484 and RGS9-2 rs1530351.

In accordance with one embodiment a patient receiving an opioid is monitored throughout the therapeutic administration of an opioid to identify any epigenetic alterations that indicate an increased risk for developing OUD. In one embodiment, the steps of monitoring a patient receiving an opioid comprises screening a DNA sample recovered from the patient to detect alterations in the methylated state of one or more genes selected from the group consisting of HOXA5, HOX6A, BLCAP, NNAT, RAI14, ZNF710, IL17B, RASSF4, ZMIZ1, ZNHIT6 and CDKAL1, or any of the intergenic regions identified in Table 1 relative to the methylated state of those genes prior to receiving the opioid treatment, or relative to a control. In one embodiment, patient is first identified as having one or more SNPs selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131 but having normal methylation of the RAI14, ZNF710, IL17B, RASSF4, ZMIZ1, ZNHIT6 and CDKAL1 genes are selected for additional monitoring for alterations in the methylated state of one or more genes or intergenic regions identified in Table 1, optionally alterations in the methylation patterns of genes selected from the group consisting of HOXA5, HOX6A, BLCAP, NNAT, RAI14, ZNF710, IL17B, RASSF4, ZMIZ1, ZNHIT6 and CDKAL1, and intergenic regions identified by methylation probes cg19636627 and cg11383134, during administration of the opioid therapy.

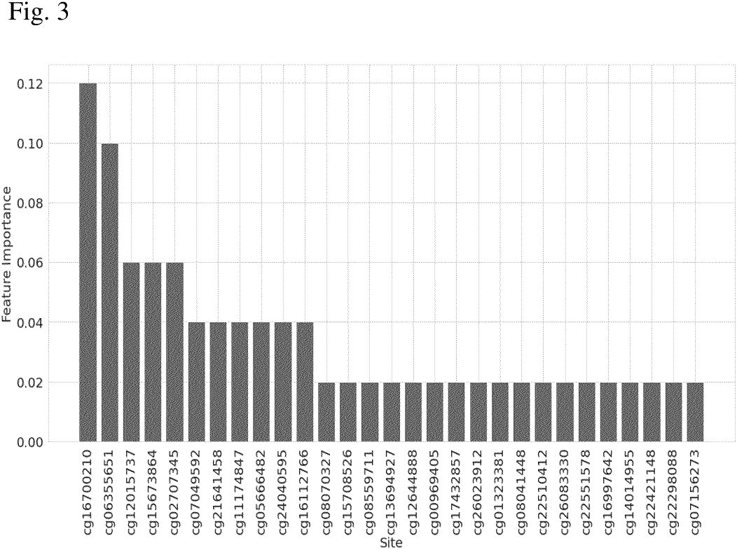

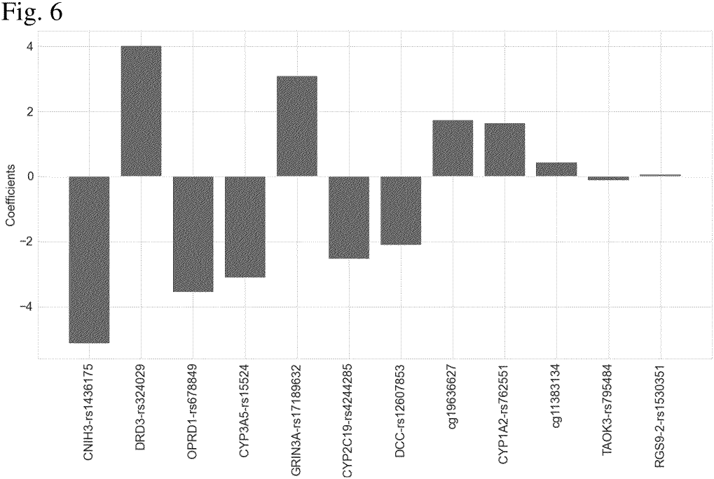

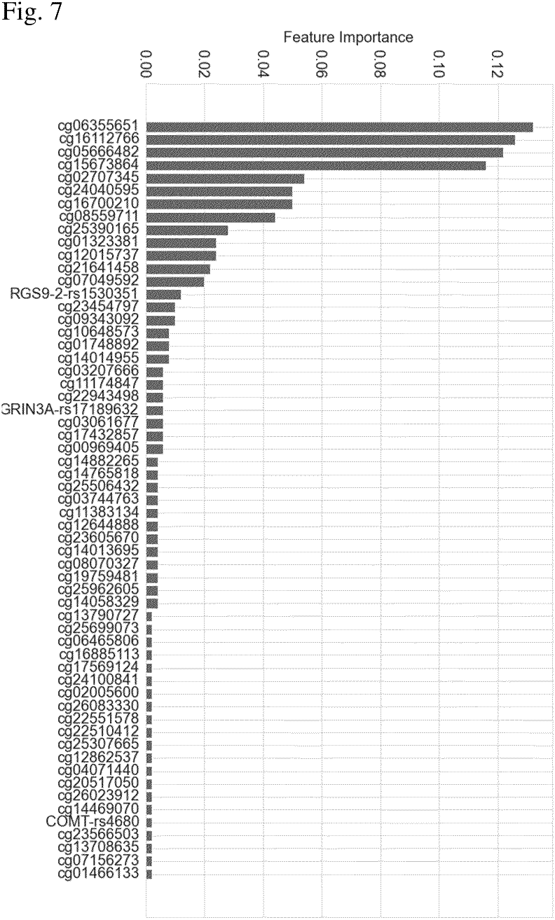

Utilizing all 103 of the probes from the differentially methylated regions and sites (see Table 1), predictive models were built with up to 95% accuracy at predicting OUD which downselected to 8 probes (LASSO model; FIG. 2) or 29 probes (Random Forest; FIG. 3). Utilizing only SNPs the predictive models were able to predict OUD with up to 85% accuracy (13 SNPs from LASSO or all 25 SNPs from Random Forest). A combinatory regression model to predict the OUD score-based methylation and SNPs was also developed with up to 95% accuracy using a total of 12 biomarkers (2 methylation sites and 10 SNPs (LASSO; FIG. 6) and a Random Forest model had 90% accuracy using 56 methylation probes and 3 SNPs (FIG. 7).

As disclosed in Example 1, analysis of methylation and SNPs in patients can differentiate those more susceptible to OUD. Methylation only models, or combined SNP and methylation models, were more accurate at predicting OUD from non-OUD compared to SNP only models (95% vs 85% accuracy). Table 2 provides a summary of some of the differentially methylated regions associated with OUD.

TABLE 2

Differentially methylated regions associated with OUD

| | | | | | |
|---|---|---|---|---|---|
| DMR | Number of Probes | Genes | Covered | Biological Relevance | |
| 1 | 42 | • | HOXA5 | • | Substance use disorder |
| | | • | HOXA6 | • | Neurodevelopmental disorders |
| 2 | 21 | • | Intergenic region between MOG | • | Pediatric obsessive |
| | | | and pseudogene SUMO2P1 | | compulsive disorder |
| 3 | 34 | • | BLCAP | • | Alcohol use |
| | | • | NNAT | • | Nicotine use |

In one embodiment SNP markers used for identification of subjects at risk of OUD, optionally in combination with one or more of the methylation markers described herein, are selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131.

In one embodiment the SNP marker used for identification of subjects at risk of OUD, optionally in combination with one or more of the methylation markers described herein (See Table 1), is selected from the group consisting of GRIN3A rs17189632, RGS9-2 rs1530351, CNIH3 rs1436175, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, BDNFOS/antiBDNF rs988712, DRD2 rs2283265, and DRD2 rs1076560. In one embodiment the SNP marker used for identification of subjects at risk of OUD, optionally in combination with one or more of the methylation markers described herein, is selected from the group consisting of CNIH3 rs1436175, OPRD1 rs678849, DRD3 rs324029, GRIN3A rs17189632, CYP2C19 rs4244285, DCC rs12607853, CYP1A2 rs762551, CYP3A5 rs15524, CYP3A5 rs776746, RGS9-2 rs1530351, ABCB1/ MDR1 rs1045642, TAOK3 rs795484, FAAH rs324420, and OPRD1 rs508448.

In one embodiment a method of screening for subjects at risk of OUD includes detecting the static makers of CNIH3 rs1436175, OPRD1 rs678849, DRD3 rs324029, GRIN3A rs17189632, CYP2C19 rs4244285, DCC rs12607853, CYP1A2 rs762551, CYP3A5 rs15524, RGS9-2 rs1530351, and TAOK3 rs795484, in combination with two differential methylation markers, optionally wherein the methylation markers are DMR2 probes 15 (cg11383134 0.7045 0.9040 0.1995 0.2883 chr6:29648590 IGR) and 20 (cg19636627 0.6307 0.8703 0.2396 0.3222 chr6:29649084 IGR).

The panel of single nucleotide polymorphisms and epigenetic markers disclosed herein can be used to determine if an individual is prone to opioid use disorder. This test could be used to determine the best course of pain management for individuals undergoing elective surgery, in the emergency department, during cancer treatment, or other medical procedure in which opioids could be prescribed.

In accordance with one embodiment a method for treating pain in subjects at risk for developing Opioid Use Disorder (OUD) is provided while minimizing the risk of OUD developing. The method comprises first screening a biological sample recovered from the subject to be administered an opioid to determine their risk for OUD. This initial screening comprises the steps of detecting the methylated state of one or more genes, or intergenic regions, identified in Table 1, optionally alterations in the methylation patterns of genes selected from the group consisting of HOXA5, HOX6A, BLCAP, NNAT, RAI14, ZNF710, IL17B, RASSF4, ZMIZ1, ZNHIT6 and CDKAL1, and intergenic regions identified by methylation probes cg19636627 and cg11383134,; and identifying individuals having two or more SNPs selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131. wherein subjects that exhibit an altered methylation pattern of two or more of those genes relative to the control (i.e. an increased methylation) and also have two or more of the above identified SNPs, are identified as having an increased risk of developing OUD relative to the general population. For those individuals identified as having an elevated risk of developing OUD, they will receive a treatment that comprises administering pain remediation therapy while avoiding opioid administration, or for those already receiving an opioid, identification of an increased risk of OUD, based on the above screening, will be switched to a different pain treatment to avoid OUD. In one embodiment the step of identifying subjects having an enhanced risk of developing OUD further comprises the step of detecting the presence of two or more of the 103 probes from the differentially methylated regions and sites identified in Table 1, anywhere from one to 25 SNP markers in a DNA sample from the subject, wherein the SNP marker is selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131. In one embodiment the step of identifying subjects having an enhanced risk of developing OUD further comprises the step of detecting the presence of SNP markers in a DNA sample from the subject, wherein the SNP marker is selected from the group consisting of CNIH3 rs1436175, OPRD1 rs678849, DRD3 rs324029, GRIN3A rs17189632, CYP2C19 rs4244285, DCC rs12607853, CYP1A2 rs762551, CYP3A5 rs15524, CYP3A5 rs776746, RGS9-2 rs1530351, ABCB1/ MDR1 rs1045642, TAOK3 rs795484, FAAH rs324420, and OPRD1 rs508448. In one embodiment the step of identifying subjects having an enhanced risk of developing OUD further comprises the step of detecting the presence of SNP markers in a DNA sample from the subject, wherein the SNP marker is selected from the group consisting of CNIH3 rs1436175, OPRD1 rs678849, DRD3 rs324029, GRIN3A rs17189632, CYP2C19 rs4244285, DCC rs12607853, CYP1A2 rs762551, CYP3A5 rs15524, RGS9-2 rs1530351, and TAOK3 rs795484.

In one embodiment the step of identifying subjects having an enhanced risk of developing OUD further comprises the step of identifying individuals having an altered methylation of two genes selected from the group consisting of RAI14, ZNF710, IL17B, RASSF4, ZMIZ1, ZNHIT6 and CDKAL1 genes relative to a control, and detecting the presence of 1-10 SNP markers in a DNA sample from the subject, wherein the SNP marker is selected from the group consisting of CNIH3 rs1436175, OPRD1 rs678849, DRD3 rs324029, GRIN3A rs17189632, CYP2C19 rs4244285, DCC rs12607853, CYP1A2 rs762551, CYP3A5 rs15524, RGS9-2 rs1530351, and TAOK3 rs795484.

The SNPs disclosed herein have been identified as inherited risk factors for OUD, while the methylation probes are biological markers for environmental influences that lead to OUD. Therefore, the diagnostic panels disclosed herein cover dynamic interactions between the genome and the environment like no other test does. This benefits the person who is being prescribed an opioid for pain management because it can be used to determine whether they should not take an opioid or if their medication should be switched to avoid further development of OUD.

In accordance with one embodiment a method of treating a subject having OUD is provided, wherein the method comprises introducing an epieffector into the cells of said subject, wherein said epieffector comprises an epigenetic regulator fused to a sequence specific endonuclease, optionally wherein the endonuclease is a dCas9, TALEN or zinc finger protein, to target removal of methylation of a gene selected from the group consisting of RAI14, ZNF710, IL17B, RASSF4, ZMIZ1, ZNHIT6 and CDKAL1 and the is an enzyme that alters methylated nucleotides. In one embodiment epigenetic regulator is a Ten-eleven translocation (TET) enzyme. Ten-eleven translocation (TET) enzymes participate in the initial step of DNA demethylation by oxidizing 5mC to 5hmC, which can lead to demethylation. Accordingly, in one embodiment the TET hydroxylase catalytic domain is linked to dCas9 to form an epieffector that targets 5mC sites on a gene selected from the group consisting of RAI14, ZNF710, IL17B, RASSF4, ZMIZ1, ZNHIT6 and CDKAL1. Such epieffectors can be introduced in to the cells of a subject to targeted DNA demethylation of genes that are associated with OUD. I one embodiment the epieffector is encapsulated or bound to a delivery system and introduced into the cells of said subject using techniques known to those skilled in the art.

In accordance with embodiment 1 a method of diagnosing a subject as being at risk for developing OUD is provided wherein the method comprises conducting a molecular assay on a DNA sample recovered from the subject, optionally wherein the DNA is isolated from a blood or tissue sample of the patient, to detect a specific panel of single nucleotide polymorphisms (SNPs) and a specific panel of methylated sites in the genome that are associated with OUD.

In accordance with embodiment 2 the method of embodiment 1 is provided wherein the methylation state of two or more genes, or intergenic regions, selected from those of Table 1 is determined wherein increased methylation of the sites identified in Table 1 identified a subject as having or at risk of developing OUD.

In accordance with embodiment 3 the method of embodiment 1 or 2 is provided wherein the panel of single nucleotide polymorphisms (SNPs) comprises two or more SNPS selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131.

In accordance with embodiment 4 the method of embodiment 2 is provided wherein the panel of SNPS comprises GRIN3A (rs17189632), RGS9-2 (rs1530351), COMT (rs4680), CNIH3 (rs1436175) and DRD2 (rs4436578).

In accordance with embodiment 5 the method of any one of embodiments 1-4 is provided wherein the panel of SNPS comprises 5-10 SNP markers.

In accordance with embodiment 6 a method of identifying a subject as being at risk of developing OUD, if two or more of the sites identified in Table 1, have elevated methylation relative to a control and two or more SNPs selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131 are detected in the patient’s biological sample.

In accordance with embodiment 7 the method of embodiment 6 is provided wherein the control represents the level of methylation of the listed sites based on average population data.

In accordance with embodiment 8 the method of embodiment 6 is provided wherein the control is based on the level of methylation of the respective genes based on detected level in individuals not susceptible to OUD.

In accordance with embodiment 9 the method of any one of embodiments 1-8 is provided wherein the subject is identified as being at risk of developing OUD, based on the identification of increased of methylation based on probes cg19636627 and cg11383134 targeting intergenic regions and the identification of SNPs CNIH3 rs1436175, DRD3 rs324029, OPRD1 rs678849, CYP3A5 rs15524, GRIN3A rs17189632, CYP2C19 rs4244285, DCC rs12607853, CYP1A2 rs762551, TAOK3 rs795484 and RGS9-2 rs1530351.

In accordance with embodiment 10 a method of detecting a set of biomarkers in the DNA of a patient is provided, said method comprising the steps of

detecting the methylated state of two or more genes selected from the group consisting of HOXA5, HOX6A, BLCAP, NNAT, RAI14, ZNF710, IL17B, RASSF4, ZMIZ1, ZNHIT6 and CDKAL1, and/or the methylated state of any of the intergenic regions identified in Table 1.

In accordance with embodiment 11 the method of embodiment 10 is provided wherein the methylated state of each of RAI14, ZNF710, IL17B, RASSF4, ZMIZ1, ZNHIT6 and CDKAL1 is determined.

In accordance with embodiment 12 the method of embodiment 10 is provided wherein the methylated state of genes CDKAL1, HOXA5 and two intergenic regions are detected using probes cg08559711 cg14014955, cg14013695, cg16997642, cg23204968, cg19643053 cg20961245 and cg18433380.

In accordance with embodiment 13 the method of any one of embodiments 10-12 is provided further comprising

detecting the presence of 1, 2, 3, 4, 5, 6, 7, 8, 9 or more SNP markers in a DNA sample from the patient wherein the SNP markers are selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846 DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131.

In accordance with embodiment 14 the method of any one of embodiments 1-13 is provided wherein the DNA sample is screened for SNP markers selected from the group consisting of GRIN3A (rs17189632), RGS9-2 (rs1530351), COMT (rs4680), CNIH3 (rs1436175) and DRD2 (rs4436578).

In accordance with embodiment 15 the method of any one of embodiments 1-13 is provided wherein the DNA sample is screened for SNP markers selected from the group consisting of CNIH3 rs1436175, DRD3 rs324029, OPRD1 rs678849, CYP3A5 rs15524, GRIN3A rs17189632, CYP2C19 rs4244285, DCC rs12607853, CYP1A2 rs762551, TAOK3 rs795484 and RGS9-2 rs1530351.

In accordance with embodiment 16 a method for treating pain in subjects at risk for developing Opioid Use Disorder (OUD) is provided, the method comprising:
identifying a subject being at risk of OUD by
   detecting the methylated state of two or more DNA sites selected from those listed in Table 1; and
   identifying subjects having an altered methylation of at least two of said DNA sites relative to a control as subjects at risk of OUD; and
avoiding opioid administration or switching to a different pain treatment to avoid OUD in the subject identified as being at risk of OUD.

In accordance with embodiment 17 the method of embodiment 16 is provided further comprising the step of

detecting the presence of SNP markers in a DNA sample from the subject, wherein the SNP marker is selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846 DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131.

In accordance with embodiment 18 the method of embodiment 16 or 17 is provided further comprising the step

detecting the presence of SNP markers in a DNA sample from the subject, wherein the SNP marker is selected from the group consisting of GRIN3A (rs17189632), RGS9-2 (rs1530351), COMT (rs4680), CNIH3 (rs1436175) and DRD2 (rs4436578).

In accordance with embodiment 19 the method of any one of embodiments 16-18 is provided wherein the methylated state of each of genes CDKAL1, HOXA5 and two intergenic regions are detected using probes cg08559711 cg14014955, cg14013695, cg16997642, cg23204968, cg19643053 cg20961245 and cg18433380.

In accordance with embodiment 20 the method of any one of embodiments 16-19 is provided wherein the SNP markers detected are selected from the group consisting of CNIH3 rs1436175, DRD3 rs324029, OPRD1 rs678849, CYP3A5 rs15524, GRIN3A rs17189632, CYP2C19 rs4244285, DCC rs12607853, CYP1A2 rs762551, TAOK3 rs795484 and RGS9-2 rs1530351.

In accordance with embodiment 21 a method of treating a subject having OUD is provided, said method comprising

introducing an epieffector into the cells of said subject, wherein said epieffector comprises an epigenetic regulator fused to a sequence specific endonuclease, to induce removal of methylation of a genomic site selected from any of those disclosed in Table 1.

In accordance with embodiment 22 the method of embodiment 21 is provided wherein the genomic DNA targeted for demethylation is a site located in a gene selected from the group consisting of HOXA5, HOX6A, BLCAP, NNAT, RAI14, ZNF710, IL17B, RASSF4, ZMIZ1, ZNHIT6 and CDKAL1.

In accordance with embodiment 23 the method of embodiment 21 or 22 is provided, wherein the endonuclease is a dCas9 or zinc finger protein and the epigenetic regulator is a ten-eleven translocation (TET) enzyme.

In accordance with embodiment 24 the method of any one of embodiments 21-23 is provided wherein the epieffector is encapsulated or bound to a delivery system and introduced into the cells of said subject.

### EXAMPLES

### Identification of Markers of OUD

The development of survey data, and single nucleotide polymorphism (SNP) data for twenty individuals, ten without OUD and ten with OUD was obtained to identify OUD biomarkers. DNA was extracted from samples obtained from the 20 individuals using Qiagen DNeasy Blood and Tissue kit spin columns. The DNA was then quantified and concentrated to meet the requirements for EPIC microarrays to detect 5mC differences between the two groups (OUD and Control). The DNA samples were then sent to Diagenode for EPIC array analysis. The raw data and a differential methylation analysis report was generated as well as genotype data for all 20 samples on 179 single nucleotide polymorphisms and survey questionnaire data for each subject.

### Results

### Survey

All 20 individuals filled out a survey upon enrollment while in the emergency room for treatment. The study participants were mostly female, and almost evenly split between Caucasian and African American. The Non-OUD Control group were on average 9 years older than the OUD group (Table 3). The OUD group had statistically significant different answers to three questions: 1. “Stop Use: Have you wanted to stop or cut down using or control your use of opioids?” 2. “Tolerance: Have you found you needed to use much more opioids to get the same effect that you did when you first started using it?” and 3. “Withdrawal: When you reduced or stopped using opioids, did you have withdrawal symptoms or felt sick when you cut down or stopped using? (aches, shaking, fever, weakness, diarrhea, nausea, sweating, heart pounding, difficulty sleeping, or feeling agitated, anxious, irritable, or depressed)? Did you use again to keep yourself from getting sick?”. These questions demonstrate opioid users with aberrant behaviors pertaining to difficulty in stopping use, withdrawal symptoms, and tolerance are more likely to be diagnosed with OUD, as these answers define OUD.

TABLE 3

Demographics

| Sample Demographics | OUD (N) | Non-OUD (N) |
|---|---|---|
| Gender | | |
| Male | 1 | 2 |
| Female | 9 | 8 |
| Age | | |
| 20-30 | 1 | - |
| 30-40 | 2 | 1 |
| 40-50 | 5 | 4 |
| 50-60 | 1 | 2 |
| 60-70 | 1 | 2 |
| >70 | - | 1 |
| Mean Age | 45 Years | 54 Years |
| Race | | |
| Black/African American | 5 | 6 |
| White | 5 | 3 |
| Prefer Not to Answer | - | 1 |
| Hispanic | | |
| Yes | - | - |
| No | 10 | 10 |
| Federal Poverty Line | | |
| Below FPL | 3 | - |
| At or Above FPL | - | 2 |
| Don’t Know/Declined to Answer | 15 | - |
| Opioid Use Disorder Severity Score | | |
| No History of Use | - | 1 |
| 0 | - | 9 |
| 4 | 2 | - |
| 6 | 1 | - |
| 7 | 1 | - |
| 9 | 1 | - |
| 11 | 5 | - |

TABLE 4

Concomitant Substance use, Family History, and Mental Health

| | OUD Group | Control Group |
|---|---|---|
| Concomitant Substance Use | | |
| Marijuana | 1 | 2 |
| Inhaled Stimulants | - | - |
| Cocaine | 2 | |
| Amphetamines | - | - |
| Hallucinogens | - | - |
| Sedatives | - | 1 |
| Alcohol | 1 | 3 |
| 1^{st} Degree Relative History of “Street” Opioid Use | | |
| Mother | - | - |
| Father | 1 | - |
| Sibling | 2 | |
| 1^{st} Degree Relative History of Prescription Opioid Use | | |
| Mother | 1 | - |
| Father | 2 | - |
| Sibling | 2 | - |
| Maternal Substance Use During Pregnancy | | |
| Yes | - | - |
| No | 1 | - |
| No response | 9 | 10 |
| Current Anxiety Symptoms (severity score > 14) | | |
| Yes | 9 | 3 |
| No | 1 | 7 |
| Current Depressive Symptoms | | |
| Yes | 6 | 2 |
| No | 4 | 8 |

TABLE 5

Item Endorsement by OUD Status

| Item | OUD | Non-OUD ^{∗} |
|---|---|---|
| Overuse: Have you often found that when you started using opioids, you ended up taking more than you intended to? For example, you planned to have a small amount of opioids but you ended up having much more; or using for a longer period than intended? | | |
| Yes | 7 | - |
| No | 3 | 9 |
| Stop Use: Have you wanted to stop or cut down using or control your use of opioids? | | |
| Yes | 9 | - |
| No | 1 | 9 |
| Time Using: Have you spent a lot of time getting or using opioids? Has it taken a lot of time for you to get over the effect? | | |
| Yes | 8 | - |
| No | 2 | 9 |
| Urges: Have you had a strong desire or urge to use opioids in between those times when you were using? Has there been a time when you had such strong urges to use that you had trouble thinking about anything else? | | |
| Yes | 8 | - |
| No | 2 | 9 |
| Life/Professional Absences: Have you missed work or school or often arrived late because you were intoxicated, high, or recovering from the night before? How about not taking care of things at home because of your use? | | |
| Yes | 5 | - |
| No | 5 | 9 |
| Relationship Problems: Has your use of opioids caused problems with other people such as with family members, friends or people at work? Do you get into arguments about your use or fights when you are using? Do you keep on using anyway? | | |
| Yes | 8 | - |
| No | 2 | 9 |
| Hobbies: Have you had to give up or spend less time working, enjoying hobbies, or being with others because of your drug use? | | |
| Yes | 6 | - |
| No | 4 | 9 |
| Coordination: Have you ever gotten high before doing something that requires coordination or concentration like driving, boating, climbing a ladder, or operating heavy machinery? Would you say that your use affected your coordination or concentration so that it was more likely that you or someone else could have been hurt? | | |
| Yes | 6 | - |
| No | 4 | 9 |
| Physical Problems: Have you continued to use even though you knew that opioids caused you problems like making you depressed, anxious, agitated, or irritable? Has your use ever caused physical problems like heart palpitations, trouble breathing or constipation? | | |
| Yes | 8 | - |
| No | 2 | 9 |
| Tolerance: Have you found you needed to use much more opioids to get the same effect that you did when you first started using it? | | |
| Yes | 10 | - |
| No | - | 9 |
| Withdrawal: When you reduced or stopped using opioids, did you have withdrawal symptoms or felt sick when you cut down or stopped using? (aches, shaking, fever, weakness, diarrhea, nausea, sweating, heart pounding, difficulty sleeping, or feeling agitated, anxious, irritable, or depressed)? Did you use again to keep yourself from getting sick? | | |
| Yes | 10 | - |
| No | - | 9 |

*1 control reported no prior opioid use & was excluded from subsequent DSM_OUD items

SNPs: Out of 179 SNPs queried, 5 were significantly different between OUD and Controls by Fisher’s Exact Test with a P-value >0.05.

64

SNPs that differentiate OUD from non-OUD controls

| Gene Symbol | NCBI SNP Reference | Function | Fisher’s Exact Test P-Value |
|---|---|---|---|
| GRIN3A | rs17189632 | NR3 subunit regulates neurotransmissions which are involved in pathophysiology and drug dependence | 0.003366602 |
| RGS9-2 | rs1530351 | involved in areas of the brain responsible for mood, motivation, and addiction; interacts with the opioid and dopamine receptor pathways | 0.005477495 |
| COMT | rs4680 | Helps break down neurotransmitters in the brain, especially active in the prefrontal cortex. Helps maintain levels of dopamine and norepinephrine. Known association with opioid addiction related to genetic changes. https://medlineplus.gov/genetics/gene/comt/ | 0.006733205 |
| CNIH3 | rs1436175 | associated with protection from pathophysiology and opioid dependence | 0.021671827 |
| DRD2 | rs4436578 | haplotype block CCGCCGTT, associated with heroin dependence | 0.024313148 |

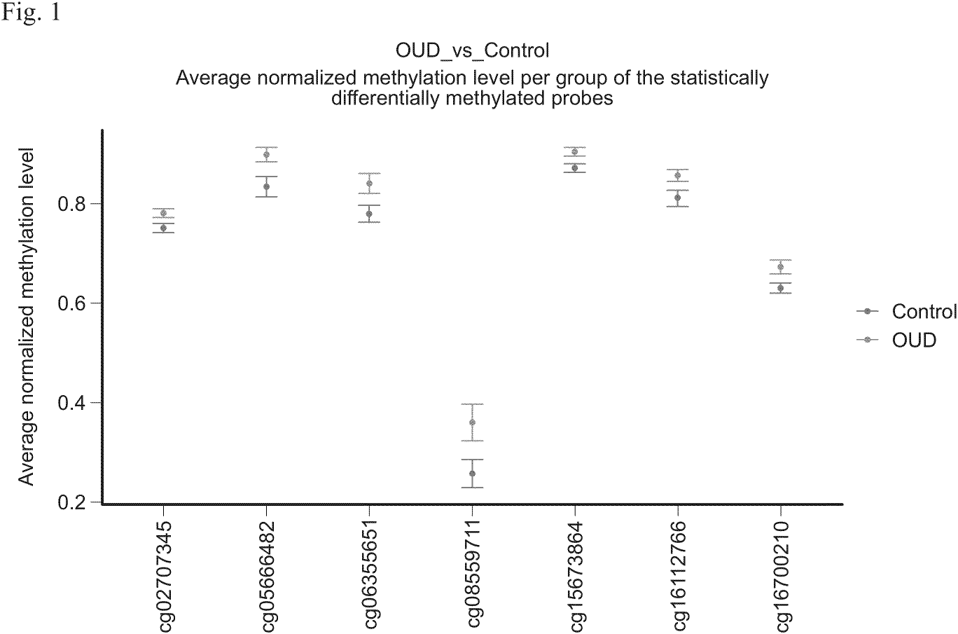

Methylation: Out of 738,372 probes queried, seven differentially methylated probes were identified (Table 6 and FIG. 1) and three differentially methylated regions (Tables 6, 7, and 8).

TABLE 6

Seven probes were significantly different between OUD and control samples

| Probe_ID | methylation difference | Adj p-value | probe position | associated gene | gene feature |
|---|---|---|---|---|---|
| cg05666482 | 0.064890 | 4.750238e-02 | chr5:34740437 | RAI14 | Body |
| cg16700210 | 0.041021 | 4.750238e-02 | chr15:9062455 | ZNF710 | 3′UTR |
| cg15673864 | 0.033000 | 4.750238e-02 | chr10:45465480 | RASSF4 | 5′UTR |
| cg16112766 | 0.046262 | 4.750238e-02 | chr10:80925057 | ZMIZ1 | 5′UTR |
| cg02707345 | 0.029543 | 4.750238e-02 | chr5:148759103 | IL17B | TSS1500 |
| cg06355651 | 0.061900 | 4.750238e-02 | chr1:86119906 | ZNHIT6 | Body |
| cg08559711 | 0.102730 | 4.750238e-02 | chr6:20978738 | CDKAL1 | Body |

Differentially methylated probes cg05666482, cg16700210, and cg02707345 from genes RAI14, ZNF710, and IL17B respectively, have not been previously reported to be associated with substance use disorders or addiction.

Differentially methylated probes cg15673864, cg16112766, and cg06355651 from genes RASSF4, ZMIZ1, and ZNHIT6 respectively, are associated with nicotine and alcohol dependence, and opioid abuse. While differentially methylated probe cg08559711 in gene CDKAL1 is associated with depressive symptoms in schizophrenic individuals.

TABLE 7

Differentially methylated Region 1

| Probe_ID | Control_average math | OUD_average math | methylation difference | Adj p-value | probe position asssociated gene | gene feature |
|---|---|---|---|---|---|---|
| cg23936031 | 0.5586 | 0.6389 | 0.0803 | 0.3670 | chr7:27183132 HOXA5 | 1stExon |
| cg09549073 | 0.4930 | 0.6195 | 0.1265 | 0.2626 | chr7:27183274 HOXA5 | 5′UTR |
| cg04863892 | 0.4917 | 0.8088 | 0.1171 | 0.4086 | chr7:27183375 HOXA5 | TSS200 |
| cg09207400 | 0.4503 | 0.5524 | 0.1022 | 0.2748 | chr7:27183382 HOXA5 | TSS200 |
| cg19739481 | 0.4994 | 0.6422 | 0.1429 | 0.3258 | chr7:27183401 HOXA5 | TSS200 |
| cg02916332 | 0.5002 | 0.6713 | 0.1110 | 0.3399 | chr7:27183591 HOXA5 | TSS1500 |
| cg17569124 | 0.4346 | 0.5682 | 0.1336 | 0.2633 | chr7:27183648 HOXA5 | TSS1500 |
| cg02005600 | 0.5659 | 0.7197 | 0.1539 | 0.2335 | chr7:27183686 HOXA5 | TSS1500 |
| cg25307663 | 0.4715 | 0.5953 | 0.1238 | 0.1903 | chr7:27183694 HOXA5 | TSS1500 |
| cg14014955 | 0.5608 | 0.7489 | 0.1861 | 0.1384 | chr7:27183701 HOXA5 | T551500 |
| cg02646423 | 0.5414 | 0.6832 | 0.1418 | 0.3335 | chr7:27183794 HOXA5 | TSS1500 |
| cg20517050 | 0.5797 | 0.7842 | 0.2044 | 0.1850 | chr7:27183806 HOXA5 | TSS1500 |
| cg23204968 | 0.6883 | 0.7960 | 0.1077 | 0.4380 | chr7:27183816 HOXA5 | TSS1500 |
| cg14058329 | 0.7772 | 0.8471 | 0.0699 | 0.2309 | chr7:27183946 HOXA5 | TSS1500 |
| cg03207656 | 0.8194 | 0.8995 | 0.0801 | 0.1819 | chr7:27183950 HOXA5 | TSS1500 |
| cg23454797 | 0.7353 | 0.8495 | 0.1143 | 0.1722 | chr7:27183990 HOXA5 | TSS1500 |
| cg12015737 | 0.8559 | 0.9103 | 0.0543 | 0.1771 | chr7:27184030 HOXA5 | TSS1500 |
| cg08070327 | 0.8262 | 0.9150 | 0.0888 | 0.2773 | chr7:27184059 HOXA5 | TSS1500 |
| cg25506432 | 0.6437 | 0.7458 | 0.1021 | 0.2642 | chr7:27184065 HOXA5 | TSS1500 |
| cg16997642 | 0.8791 | 0.9425 | 0.0634 | 0.3288 | chr7:27184159 HOXA5 | TSS1500 |
| cg20817131 | 0.6785 | 0.7905 | 0.1120 | 0.3461 | chr7:27184167 HOXA5 | TSS1500 |
| cg14013695 | 0.8110 | 0.8662 | 0.0558 | 0.2917 | chr7:27184176 HOXA5 | TSS1500 |
| cg25390165 | 0.6500 | 0.7794 | 0.1294 | 0.1003 | chr7:27184188 HOXA5 | TSS1500 |
| cg01323381 | 0.6283 | 0.7635 | 0.1351 | 0.1706 | chr7:27184264 HOXA5 | TSS1500 |
| cg19643053 | 0.8291 | 0.8798 | 0.0507 | 0.3432 | chr7:27184271 HOXA5 | TSS1500 |
| cg26023912 | 0.5483 | 0.6794 | 0.1311 | 0.1796 | chr7:27184369 HOXA5 | TSS1500 |
| cg14882265 | 0.5915 | 0.7208 | 0.1292 | 0.1565 | chr7:27184375 HOXA5 | TSS1500 |
| cg17432857 | 0.5323 | 0.6749 | 0.1426 | 0.2313 | chr7:27184438 HOXA5 | TSS1500 |
| cg00969405 | 0.6276 | 0.7740 | 0.1465 | 0.1954 | chr7:27184441 HOXA5 | TSS1500 |
| cg07049592 | 0.6471 | 0.7609 | 0.1138 | 0.0960 | chr7:27184450 HOXA5 | TSS1500 |
| cg02106682 | 0.5563 | 0.6496 | 0.0933 | 0.2781 | chr7:27184461 HOXA5 | TSS1500 |
| cg03368099 | 0.5093 | 0.5861 | 0.0768 | 0.3261 | chr7:27184521 HOXA5 | TSS1500 |
| cg01748892 | 0.3955 | 0.4306 | 0.0351 | 0.3559 | chr7:27184667 HOXA5 | TSS1500 |
| cg13694927 | 0.4097 | 0.4430 | 0.0333 | 0.5209 | chr7:27184712 HOXA5 | TSS1500 |
| cg03744763 | 0.2578 | 0.3334 | 0.0756 | 0.3047 | chr7:27184737 HOXA5 | TSS1500 |
| cg27151303 | 0.4228 | 0.5178 | 0.0950 | 0.2815 | chr7:27184821 | IGR |
| cg05579037 | 0.6551 | 0.7573 | 0.1022 | 0.3130 | chr7:27184853 | IGR |
| cg21641458 | 0.6788 | 0.7873 | 0.1086 | 0.1279 | chr7:27185136 HOXA6 | 3′UTR |
| cg09343092 | 0.6828 | 0.7596 | 0.0768 | 0.1105 | chr7:27185282 HOXA6 | Body |
| cg24040595 | 0.7475 | 0.8618 | 0.1141 | 0.1180 | chr7:27185512 HOXA6 | Body |
| cg08465806 | 0.7614 | 0.8558 | 0.0942 | 0.2314 | chr7:27185732 HOXA6 | Body |

Differentially methylated region HOXA5 has been associated with substance use disorder and differentially methylated region HOXA6 has been associated with neurodevelopmental disorders.

TABLE 8

Differentially methylated Region 2

| Probe_ID | Control_average meats | OUD_average meth | methylation difference | Adj p-value | probe position | associated gene | gene feature |
|---|---|---|---|---|---|---|---|
| cg25878138 | 0.8584 | 0.8587 | 8.1083 | 0.3448 | chr6:28648161 | | IGR |
| cg1747584 | 0.6948 | 0 8796 | 0.1848 | 0.3476 | chr8.28648226 | | IGR |
| cg15708626 | 0.7880 | 0.8892 | 8.1282 | 0.3688 | chr6.28648271 | | IGR |
| cg04871440 | 0.7127 | 0.8467 | 0.1348 | 0.2825 | chr6.28648276 | | IGR |
| cg08822281 | 0.6844 | 3.6005 | 0.0981 | 0.2811 | chr6.28648345 | | IGR |
| cg10648373 | 0.5321 | 0.6418 | 0.1087 | 0.2382 | chr6:29648348 | | IGR |
| cg12641888 | 0.8410 | 0.6524 | 0.1114 | 0.3341 | chr6:29648380 | | IGR |
| cg22404832 | 0.7750 | 0.9358 | 0.1808 | 0.3603 | chr:20648379 | | IGR |
| cg25690078 | 0.7878 | 0.9849 | 0.1470 | 0.3284 | chr6:28848381 | | IGR |
| cg07134866 | 0.7217 | 0.8978 | 0.1761 | 0.4094 | chr6:29648400 | | IGR |
| cg00588198 | 0.5799 | 0.8003 | 0.2298 | 0.2409 | chr6:29648452 | | IGR |
| cg06082337 | 0.2126 | 0.3616 | 0.1300 | 0.6803 | chr6.28648468 | | IGR |
| cg16885113 | 0.7445 | 0.9168 | 0.1714 | 0.3883 | chr6:28648507 | | IGR |
| cg20228636 | 0.6600 | 0.8740 | 0.2089 | 0.2633 | chr3:28648625 | | IGR |
| cg11383134 | 0.7045 | 0.9040 | 0.1995 | 0.2883 | chr6:29648500 | | IGR |
| cg03449867 | 0.6872 | 0.8722 | 8.1850 | 0.4188 | chr6:29648623 | | IGR |
| cg16570650 | 0.0795 | 0.8814 | 0.2010 | 0.3020 | chr6:29648628 | | IGR |
| cg08041448 | 0.4681 | 0.6236 | 0.1655 | 0.4815 | chr6:29648801 | | IGR |
| cg24100641 | 0.5500 | 0.7810 | 0.2310 | 0.3469 | chr6:29648824 | | IGR |
| cg19636627 | 0.6307 | 0.8703 | 0.2336 | 0.3222 | chr6:29649884 | | IGR |
| cg20361245 | 0.4563 | 0.7272 | 0.2710 | 0.6029 | chr6:29648092 | | IGR |

The intergenic region of chromosome 6 29633374-29668117 includes part of gene MOG and pseudogene SUMO2P1 and was identified as a differentially methylated region. This region is associated with pediatric obsessive compulsive disorder.

TABLE 9

Differentially methylated Region 3

| Probe_ID | Control_average meth | OUD_average meth | methylation difference | Adj p-value | probe position | associated gene | gene feature |
|---|---|---|---|---|---|---|---|
| cg23767721 | 0.6284 | 0.7273 | 0.0878 | 0.2313 | chr20:36148604 | BLCAP | SUTR |
| cg26083330 | 0.6731 | 0.7296 | 0.0663 | 0.2830 | chr20:36148615 | BLCAP | SUTR |
| cg22551578 | 0.5151 | 0.5918 | 0.0768 | 0.2936 | chr20:36148620 | BLCAP | SUTR |
| cg04489586 | 0.6108 | 0.8698 | 0.0531 | 0.2584 | chr20:36148642 | BLCAP | SUTR |
| cg23603670 | 0.5467 | 0.5081 | 0.0513 | 0.4245 | chr20:36148672 | BLCAP | SUTR |
| cg12862537 | 0.5130 | 0.8016 | 0.0886 | 0.3497 | chr20:36148679 | BLCAP | SUTR |
| cg10981698 | 0.5570 | 0.6047 | 0.0477 | 0.4977 | chr20:36148698 | BLCAP | SUTR |
| eg13790727 | 0.6835 | 0.7392 | 0.0557 | 0.3080 | chr20:36148738 | BLCAP | SUTR |
| cg28783699 | 0.8612 | 0.7164 | 0.0531 | 0.4381 | chr20:36148767 | BLCAP | SUTR |
| cg23712981 | 0.6974 | 0.7470 | 0.0496 | 0.4130 | chr20:36148775 | BLCAP | SUTR |
| cg03615235 | 0.6888 | 0.7495 | 0.0697 | 0.3770 | chr20:86148779 | BLCAP | SUTR |
| cg17643825 | 0.7172 | 0.7786 | 0.0814 | 0.4134 | chr20:36146781 | BLCAP | SUTR |
| cg24762053 | 0.6344 | 0.7251 | 0.0707 | 0.3763 | chr20:36148798 | BLCAP | SUTR |
| cg4469070 | 0.6952 | 0.7705 | 0.0723 | 0.4211 | chr20:36148803 | BLCAP | SUTR |
| eg22298088 | 0.6240 | 0.7220 | 0.0980 | 0.3332 | chr20:36148860 | BLCAP | SUTR |
| cg22943498 | 0.6324 | 0.7116 | 0.0791 | 0.3526 | chr20^{:}36148028 | BLCAP | SUTR |
| cg14765818 | 0.5641 | 0.6214 | 0.0573 | 0.2663 | chr20:36148954 | BLCAP | SUTR |
| cg16473473 | 0.5484 | 0.6579 | 0.1094 | 0.2683 | chr2036148994 | BLCAP | SUTR |
| cg07156273 | 0.6809 | 0.6817 | 0.1009 | 0.2931 | chr20:36149013 | BLCAP | SUTR |
| cg01466133 | 0.5916 | 0.8947 | 0.1031 | 0.3133 | chr2036149022 | BLCAP | SUTR |
| cg24675557 | 0.5913 | 0.8732 | 0.0819 | 0.2950 | chr20:36148081 | BLCAP | SUTR |
| cg20479680 | 0.5547 | 0.6514 | 0.0968 | 0.2987 | chr20:36149112 | BLCAP | SUTR |
| cg22421148 | 0.5850 | 0.6800 | 0.0949 | 0.2880 | chr20:36149119 | BLCAP | SUTR |
| cg22510412 | 0.6126 | 0.72336 | 0.1209 | 0.2462 | chr20:36149121 | BLCAP | SUTR |
| cg03061677 | 0.6006 | 0.6506 | 0.0899 | 0.2655 | chr20:36149185 | BLCAP | SUTR |
| cg00576435 | 0.5449 | 0.6382 | 0.0933 | 0.2519 | chr20:36149188 | BLCAP | SUTR |
| cg18433380 | 0.5266 | 0.6414 | 0.1148 | 0.2482 | chr20:36149194 | BLCAP | SUTR |
| cg16648571 | 0.5015 | 0.6677 | 0.0661 | 0.2840 | chr20:36149231 | BLCAP | SUTR |
| cg04810287 | 0.5262 | 0.6342 | 0.1080 | 0.3253 | chr20:36149271 | BLCAP | SUTR |
| cg25962605 | 0.4800 | 0.5538 | 0.0728 | 0.2745 | chr20:361149452 | BLCAP | SUTR |
| cg21588305 | 0.5835 | 0.6806 | 0.0971 | 0.2154 | chr20:36149465 | BLCAP | SUTR |
| cg13708636 | 0.4964 | 0.5270 | 0.0306 | 0.4438 | chr20:36149656 | NNAT | SUTR |
| cg11174847 | 0.5316 | 0.5784 | 0.468 | 0.2223 | chr20:36149706 | NNAT | SUTR |
| cg23566503 | 0.5068 | 0.5612 | 0.0545 | 0.2711 | chr20:36149750 | NNAT | 1stExon |

The gene BLCAP in differentially methylated region 3 has been shown to affect methylation signatures following alcohol use. The NNAT gene in differentially methylated region 3 is associated with nicotine use in rats.

### Methylation Based OUD Prediction

Three different modeling approaches were used to build models to predict OUD using the significantly different probes and differentially methylated regions: 1. Decision Tree, 2. LASSO, and 3. Random Forest. The decision tree model had the lowest accuracy at 0.75, using only one probe to differentiate OUD from non-OUD. After parameter tuning, both the LASSO and Random Forest models had an accuracy of 0.95. The LASSO model utilized 8 probes, while the Random Forest model utilizes 29 probes. Comparing the features used in the three models, the Decision tree chosen feature was also identified as important by Random Forest, and three of the 8 probes identified by LASSO were also chosen by the Random Forest model.

### SNP Based OUD Prediction

As with the methylation-based OUD prediction, the same three machine learning models were used to predict OUD using the top 25 SNPs that differentiate OUD from non-OUD controls. The decision tree and LASSO models tied for the lowest accuracy at 75%, while the random forest had an accuracy of 85%. After tuning, the LASSO model accuracy increased to 85% while the others decreased performance slightly or stayed the same. Comparing the features used in the three models, two of the chosen SNPs used by the decision tree were also chosen by the LASSO and random forest models. The random forest model used all the SNPs, while LASSO used 13 SNPs.

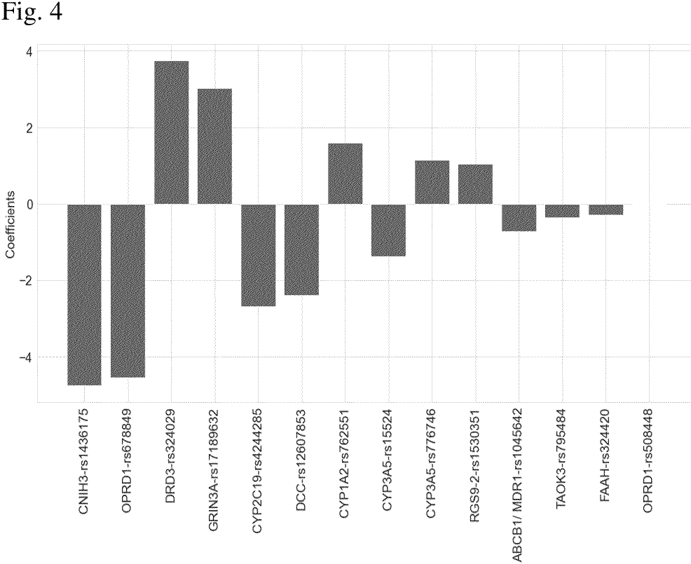

FIG. 4. The LASSO model utilized 13 SNPs to differentiate OUD from controls.

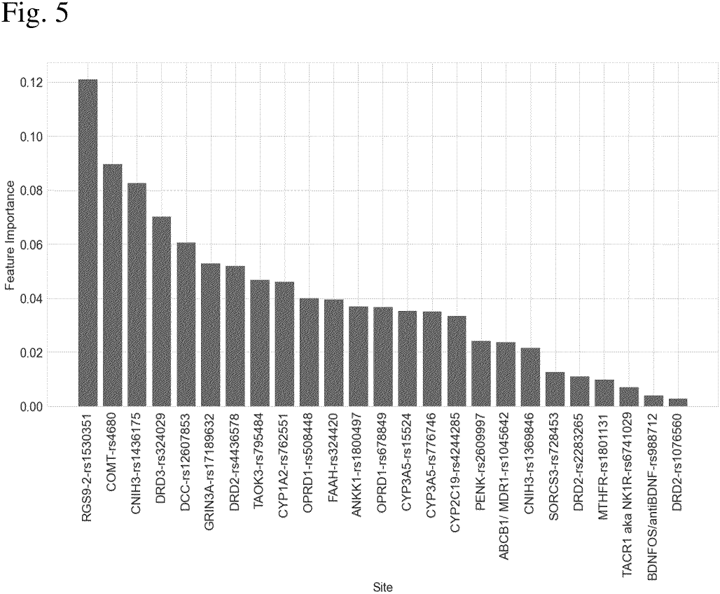

FIG. 5. The random forest model utilized all 25 SNPs to differentiate OUD from controls.

### Combined OUD Prediction

We used the same machine learning models in the previous analyses to predict OUD after combining the methylation and SNP data. Before tuning the LASSO, decision tree, and random forest models had 60%, 85% and 90% accuracy, respectively. After tuning, the LASSO model saw the most improvement with 95% accuracy while the performance for the decision tree and random forest models stayed the same. The decision tree used 1 methylation probe (cg16112766), while LASSO used 2 methylation probes and 10 SNPs. The random forest used 56 methylation probes and 3 SNPs.

FIG. 6. LASSO model used a mix of methylation probes (2) and SNPs (10) to differentiate OUD from controls.

FIG. 7. Random forest used a mix of methylation probes (56) and SNPs (3) to differentiate OUD from controls.

### CONCLUSIONS

The decision tree models generally perform the worst out of the three models and they tend to pick the smallest number of features to use. The random forest models have comparable performance to the LASSO model, but tend to pick too many features. The LASSO models have the best performance across each of the three (methylation, SNP, combined) datasets while using the fewest probes and SNPs to obtain those results.

1. A method of diagnosing a subject as being at risk for developing OUD, said method comprising conducting a molecular assay on a DNA sample recovered from the subject to detect a specific panel of single nucleotide polymorphisms (SNPs) and a specific panel of methylated sites in the genome that are associated with OUD.

2. The method of claim 1 wherein the methylation state of two or more genes, or intergenic regions, selected from the sites identified by DMR probe 1, DMR probe 2, DMR probe 3, DMR probe 4, DMR probe 5, DMR probe, DMR probe 7, DMR probe 8, DMR probe 9, DMR probe 10, DMR probe 11, DMR probe 12, DMR probe 13, DMR probe 14, DMR probe 15, DMR probe 16, DMR probe 17, DMR probe 18, DMR probe 19, DMR probe 20, DMR probe 21, DMR probe 22, DMR probe 23, DMR probe 24, DMR probe 25, DMR probe 26, DMR probe 27, DMR probe 28, DMR probe 29, DMR probe 30, DMR probe 31, DMR probe 32, DMR probe 33, DMR probe 34, DMR probe 35, DMR probe 36, DMR probe 37, DMR probe 38, DMR probe 39, DMR probe 40, DMR probe 41, DMR 2 probe 1, DMR 2 probe 2, DMR 2 probe 3, DMR 2 probe 4, DMR 2 probe 5, DMR 2 probe 6, DMR 2 probe 7, DMR 2 probe 8, DMR 2 probe 9, DMR 2 probe 10, DMR 2 probe 11, DMR 2 probe 12, DMR 2 probe 13, DMR 2 probe 14, DMR 2 probe 15, DMR 2 probe 16, DMR 2 probe 17, DMR 2 probe 18, DMR 2 probe 19, DMR 2 probe 20, DMR 2 probe 21, DMR 3 probe 1, DMR 3 probe 2, DMR 3 probe 3, DMR 3 probe 4, DMR 3 probe 5, DMR 3 probe 6, DMR 3 probe 7, DMR 3 probe 8, DMR 3 probe 9, DMR 3 probe 10, DMR 3 probe 11, DMR 3 probe 12, DMR 3 probe 13, DMR 3 probe 14, DMR 3 probe 15, DMR 3 probe 16, DMR 3 probe 17, DMR 3 probe 18, DMR 3 probe 19, DMR 3 probe 20, DMR 3 probe 21, DMR 3 probe 22, DMR 3 probe 23, DMR 3 probe 24, DMR 3 probe 25, DMR 3 probe 26, DMR 3 probe 27, DMR 3 probe 28, DMR 3 probe 29, DMR 3 probe 30, DMR 3 probe 31, DMR 3 probe 32, DMR 3 probe 33, or DMR 3 probe 34, is determined wherein increased methylation of two or more sites identified by said DMR probes identifies a subject as having or at risk of developing OUD.

3. The method of claim 2 wherein
the panel of single nucleotide polymorphisms (SNPs) comprises two or more SNPS selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131.

4. The method of claim 3 wherein the panel of SNPS comprises GRIN3A (rs17189632), RGS9-2 (rs1530351), COMT (rs4680), CNIH3 (rs1436175) and DRD2 (rs4436578).

5. A method of identifying a subject as being at risk of developing OUD, if two or more of the sites of claim 2 have elevated methylation relative to a control and two or more SNPs selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846, DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131 are detected in the patient’s biological sample.

6. The method of claim 5 wherein
i) the control represents the level of methylation of the listed sites based on average population data; or
ii) the control is based on the level of methylation of the respective genes based on detected level in individuals not susceptible to OUD.

7. The method of claim 6 wherein the subject is identified as being at risk of developing OUD, based on the identification of increased of methylation based on probes cg19636627 (SEQ ID NO: 9) and cg11383134 (SEQ ID NO: 8) targeting intergenic regions and the identification of SNPs CNIH3 rs1436175, DRD3 rs324029, OPRD1 rs678849, CYP3A5 rs15524, GRIN3A rs17189632, CYP2C19 rs4244285, DCC rs12607853, CYP1A2 rs762551, TAOK3 rs795484 and RGS9-2 rs1530351.

8. A method of detecting a set of biomarkers in the DNA of a patient, said method comprising the steps of
detecting the methylated state of two or more genes selected from the group consisting of HOXA5, HOX6A, BLCAP, NNAT, RAI14, ZNF710, IL17B, RASSF4, ZMIZ1, ZNHIT6 and CDKAL1, and/or the methylated state of any of the intergenic regions identified in claim 2, optionally wherein the methylated state of each of RAI14, ZNF710, IL17B, RASSF4, ZMIZ1, ZNHIT6 and CDKAL1 is determined.

9. The method of claim 8 wherein the methylated state of genes CDKAL1, HOXA5 and two intergenic regions are detected using probes cg08559711 cg14014955, cg14013695, cg16997642, cg23204968, cg19643053 cg20961245 and cg18433380.

10. The method of claim 9 further comprising
detecting the presence of SNP markers in a DNA sample from the patient wherein the SNP markers are selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846 DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131, optionally wherein the SNP markers are selected from the group consisting of GRIN3A (rs17189632), RGS9-2 (rs1530351), COMT (rs4680), CNIH3 (rs1436175) and DRD2 (rs4436578).

11. The method of claim 8 wherein the SNP markers are selected from the group consisting of CNIH3 rs1436175, DRD3 rs324029, OPRD1 rs678849, CYP3A5 rs15524, GRIN3A rs17189632, CYP2C19 rs4244285, DCC rs12607853, CYP1A2 rs762551, TAOK3 rs795484 and RGS9-2 rs1530351.

12. A method for treating pain in subjects at risk for developing Opioid Use Disorder (OUD), the method comprising:
identifying a subject being at risk of OUD by
detecting the methylated state of two or more DNA sites selected from those listed in claim 2; and
identifying subjects having an altered methylation of at least two of said DNA sites relative to a control as subjects at risk of OUD; and
avoiding opioid administration or switching to a different pain treatment to avoid OUD in the subject identified as being at risk of OUD.

13. The method of claim 12 further comprising the step of
detecting the presence of SNP markers in a DNA sample from the subject, wherein the SNP marker is selected from the group consisting of GRIN3A rs17189632, RGS9-2 rs1530351, COMT rs4680, CNIH3 rs1436175, DRD2 rs4436578, OPRD1 rs508448, CYP2C19 rs4244285, OPRD1 rs678849, CYP3A5 rs15524, CYP3A5 rs776746, TAOK3 rs795484, ANKK1 rs1800497, DRD3 rs324029, SORCS3 rs728453, CNIH3 rs1369846 DCC rs12607853, PENK rs2609997, TACR1 aka NK1R rs6741029, CYP1A2 rs762551, FAAH rs324420, ABCB1/ MDR1 rs1045642, BDNFOS/antiBDNF rs988712, DRD2 rs2283265, DRD2 rs1076560 and MTHFR rs1801131.

14. The method of claim 12 further comprising the step
detecting the presence of SNP markers in a DNA sample from the subject, wherein the SNP marker is selected from the group consisting of GRIN3A (rs17189632), RGS9-2 (rs1530351), COMT (rs4680), CNIH3 (rs1436175) and DRD2 (rs4436578).

15. The method of claim 13 wherein the methylated state of each of genes CDKAL1, HOXA5 and two intergenic regions are detected using probes cg08559711 cg14014955, cg14013695, cg16997642, cg23204968, cg19643053 cg20961245 and cg18433380.

16. The method of claim 15 wherein the SNP markers detected are selected from the group consisting of CNIH3 rs1436175, DRD3 rs324029, OPRD1 rs678849, CYP3A5 rs15524, GRIN3A rs17189632, CYP2C19 rs4244285, DCC rs12607853, CYP1A2 rs762551, TAOK3 rs795484 and RGS9-2 rs1530351.

17. A method of treating a subject having OUD, said method comprising
introducing an epieffector into the cells of said subject, wherein said epieffector comprises an epigenetic regulator fused to a sequence specific endonuclease, to induce removal of methylation of a genomic site selected from any of those disclosed in claim 2.

18. The method of claim 17 wherein the genomic DNA targeted for demethylation is a site located in a gene selected from the group consisting of HOXA5, HOX6A, BLCAP, NNAT, RAI14, ZNF710, IL17B, RASSF4, ZMIZ1, ZNHIT6 and CDKAL1.

19. The method of claim 17, wherein the endonuclease is a dCas9 or zinc finger protein and the epigenetic regulator is a ten-eleven translocation (TET) enzyme.

20. The method of claim 17 wherein the epieffector is encapsulated or bound to a delivery system and introduced into the cells of said subject.
